(19) United States
(12) Patent Application Publication
(10) Pub. No.: US 2014/0249839 A1
(43) Pub. Date: Sep. 4, 2014

(54) METHOD AND SYSTEM RELATING TO A LABORATORY PRODUCT

(71) Applicant: Cable Software (Dental) Limited, London (GB)

(72) Inventors: Marcus RICKARD, London (GB); John WILLMOTT, Nottingham (GB); Rick CUSIMANO, Sheffield (GB)

(73) Assignee: Cable Software (Dental) Limited, London (GB)

(21) Appl. No.: 14/194,450

(22) Filed: Feb. 28, 2014

(30) Foreign Application Priority Data
Mar. 1, 2013 (GB) 1303727.0

(51) Int. Cl. G06Q 10/08; G06Q 50/22

(52) U.S. Cl. CPC G06Q 10/087; G06Q 50/22
USPC 705/2

(57) ABSTRACT

A method of providing data indicative of options for supply of a dental laboratory product is disclosed. The method may include receiving data indicative of the dental laboratory product and based on the received data, retrieving data indicative of a first value of a parameter of the dental laboratory product and a second value of the parameter of the dental laboratory product. Based on a comparison of the generated values, data is generated to allow for selection of one of the products.

### CROSS-REFERENCE TO RELATED APPLICATIONS

This application claims priority under 35 U.S.C. §119(a) to GB Application No. GB1303727.0, filed Mar. 1, 2013, the entire content of which is hereby incorporated by reference.

### BACKGROUND OF THE INVENTION

1. Field of the Invention

This application relates to a method and system relating to a laboratory product, particularly, but not exclusively, to a dental laboratory product.

2. Description of the Related Technology

The present invention relates to an improved system for use in the provision of a dental laboratory product.

### SUMMARY

According to a first aspect of the present invention, there is provided a method of providing data indicative of options for supply of a dental laboratory product, the method including:

a) receiving data indicative of the dental laboratory product;

b) on the basis of said data received in step a), retrieving from a data store, or generating, data indicative of a first value of a parameter of the dental laboratory product;

c) on the basis of said data received in step a), retrieving from a data store, or generating, data indicative of a second value of the parameter of the dental laboratory product; and

d) generating data representative of a comparison of the first value with the second value.

The present invention therefore allows a comparison to be made of different values, for example a first and a second value, of a dental laboratory product parameter. As a dental laboratory product may be custom made for a patient, and is therefore unique, and moreover as, for example, different suppliers of dental laboratory products may provide information on the supply of a dental laboratory product in a different format, the present invention allows a comparison to be made to allow for example a dentist to choose the preferred option for supply of a dental laboratory product.

Generating the data in step d) may include comparing the first value with the second value according to a comparison criteria, and generating data indicative of a preferred option for supply of the dental laboratory product.

The data generated in step d) may be representative of a list comprising the first and second values.

The comparison criteria may comprise identifying the lowest of the first and second value.

The parameter may be price, and optionally the first value relates to a first dental laboratory product supplier and the second value relates to a different, second dental laboratory product supplier.

The data received in step a) may comprise data indicating a dental laboratory product type.

The dental laboratory product type may be selected from the group consisting of: a jaw product, a tooth product and a bridge product.

The dental laboratory product type may relate to one of: an upper jaw position, a lower position, any jaw position, an anterior tooth position, a premolar tooth position, a molar tooth position, a combination of any of the aforesaid tooth positions, any tooth position, a pontic bridge type, a retainer bridge type and any bridge type.

The retrieved data indicative of the first value may have been, or the data indicative of the first value may be, generated using a first calculation algorithm to convert data indicative of the first value from a first data format to a standard data format, and the retrieved data indicative of the second value has been, or the data indicative of the second value is, generated using a different, second calculation algorithm to convert data indicative of the second value from a different, second data format to the standard data format.

This allows data indicative of a parameter value which is provided in different data formats by, for example, different dental laboratories, to be converted to a standard data format, thereby allowing for example a comparison to be made between different values, e.g. a first and a second value, of a dental laboratory product supplied by different laboratories.

The method may comprise: selecting a dental laboratory for supply of the dental laboratory product, on the basis of the comparison in d); converting, using a calculation algorithm, data indicative of a value of the dental laboratory product from a standard data format to a different data format recognizable by the selected dental laboratory, and optionally transmitting data indicative of the dental laboratory product to the selected dental product, the transmitted data including the value in said different data format.

This allows a dental laboratory product to be selected for order, from the selected dental laboratory. For the order to be placed with the selected dental laboratory, a value of a parameter of the dental laboratory product may be converted from the standard data format, such as the standard data format referred to previously, to a different data format recognizable by a system of the dental laboratory selected and with which the order is to be placed. Thus, a dental laboratory product may be ordered easily.

The method may include using said data indicative of a preferred option for supply of the dental laboratory product to identify a preferred supplier of the dental laboratory product.

The method may include:

i) on the basis of said data received in step a), retrieving from a data store or generating data indicative of a first value of at least one further parameter of the dental laboratory product;

ii) on the basis of said data received in step a), retrieving from a data store or generating data indicative of a second value of the at least one further parameter of the dental laboratory product,

said generating the data indicative of the preferred option for supply of the dental laboratory product including comparing the first value with the second value of the at least one further parameter.

The at least one further parameter may include at least one of a quality of a dental laboratory product from a supplier, a delivery time of a dental laboratory product from a supplier, and a delivery area of a dental laboratory product supplier.

Said generating the data indicative of the preferred option may include preferentially weighting one of the parameter and the at least one further parameter such that the data indicative of the preferred option is generated according to the preferential weighting.

The data received in step a) may include data identifying the dental laboratory product.

The dental laboratory product may be a custom-made product for a patient.

According to a second aspect of the invention there is provided a method of generating data indicative of a parameter of a dental laboratory product, the method including:

generating a first value of the parameter of the dental laboratory product using a first calculation algorithm to convert data indicative of the first value from a first data format to a standard data format,

generating a second value of the parameter of the dental laboratory product using a different, calculation algorithm to convert data indicative of the second value from a different, second data format to the standard data format.

This allows data indicative of a parameter value which is provided in different data formats by, for example, different dental laboratories, to be converted to a standard data format, thereby allowing for example a comparison to be made between different values, e.g. a first and a second value, of a dental laboratory product supplied by different laboratories.

The parameter may be price.

The first data format may be provided by a first supplier of the dental laboratory product and the second data format is provided by a different, second supplier of the dental laboratory product.

According to a third aspect of the invention, there is provided a system arranged to perform a method in accordance with any of the examples of the present invention described.

According to a fourth aspect of the invention, there is provided a computer program comprising computer readable instructions which, when executed, provides a method according to the invention.

According to a fifth aspect of the invention, there is provided a computer program product comprising a non-transitory computer-readable storage medium having computer readable instructions stored thereon, the computer readable instructions being executable by a computerized device to cause the computerized device to perform a method in accordance with the invention.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1 shows schematically an example of the first aspect of the invention.

FIG. 2 shows schematically an example of the second aspect of the invention.

FIG. 3 shows schematically an example of a system comprising parts for providing the first and second aspects of the invention.

FIG. 4 shows schematically an example method of classifying a dental laboratory product to generate a value of a price parameter.

FIGS. 5 to 9 illustrate an example of placing an order for a dental laboratory product.

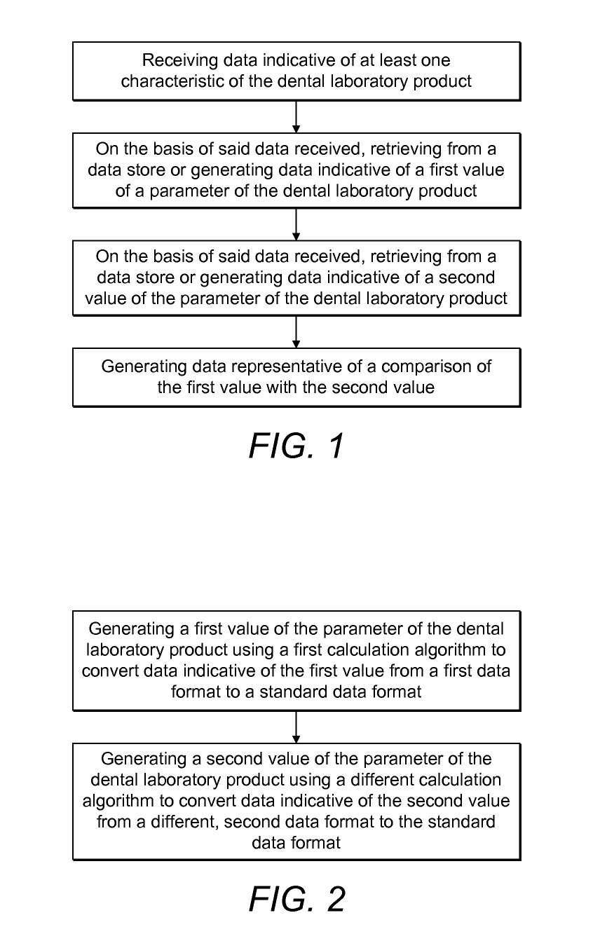

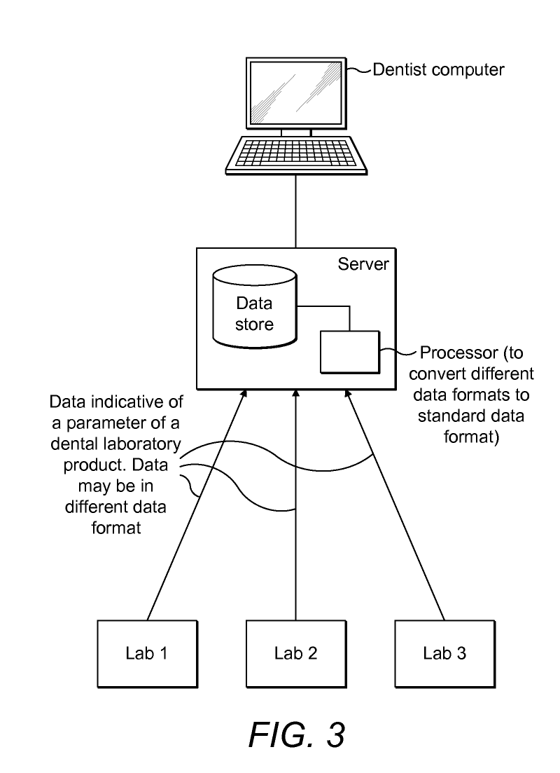

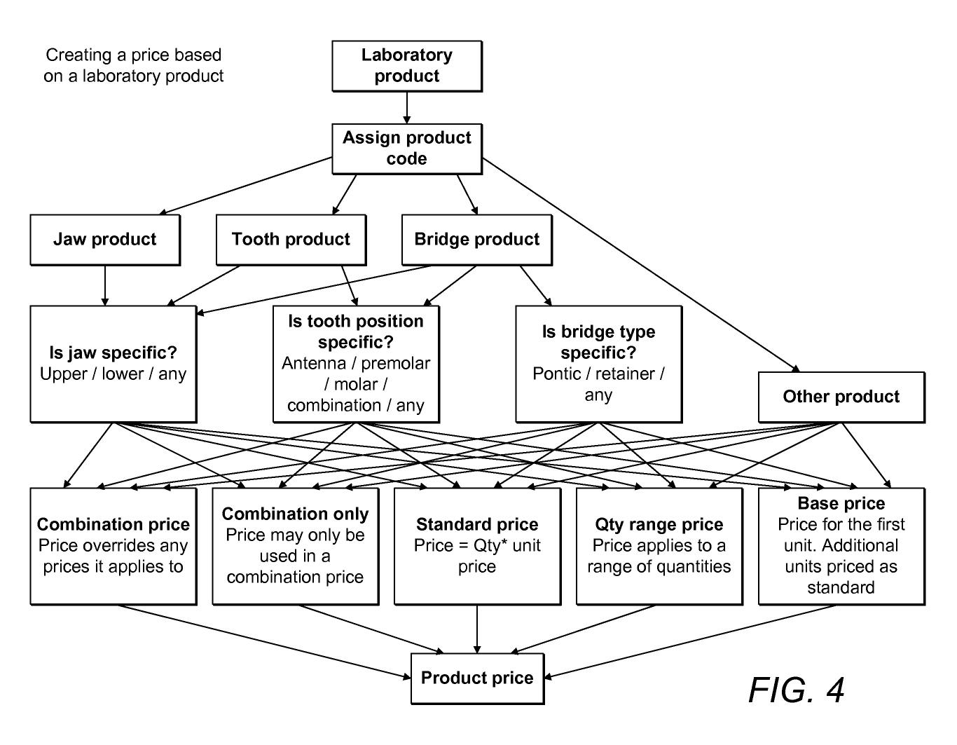

### DETAILED DESCRIPTION OF CERTAIN INVENTIVE EMBODIMENTS

According to an example of a system of the present invention, there is provided custom made cloud based software that has two primary functions: (1) to provide comprehensive ‘clinical’ governance over the relationships between the dentist/s and/or dental group/s and the dental laboratories suppliers that service the dental market in the UK (and around the world) with custom made dental laboratory products, colloquially referred to as “dental laboratory work”; and (2) to enable dentist/s and/or dental group/s to compare dental laboratory products between dental laboratory suppliers based on a ‘bespoke search criteria’ designed so as to enable the selection and purchase orders of crown and bridge work for the first time on an informed basis and importantly in a more cost-effective manner.

A coding table facilitates the comparison of the many configurations and descriptions of products that are provided by any dental laboratory. This together with the way that software according to the invention handles these elements is the engine of our software, allowing true comparisons to be made across an amazing range of product descriptions.

EXAMPLE PRODUCT CODES FROM THE CODING TABLE:
Veneers (1600-1699) (100 Codes)
1600 e.max Veneer
1605 3600 Veneer
1610 Zirconium Veneer
1615 Porcelain Veneer
1620 Re-etch Veneer
1630 Composite Veneer

### System Setup

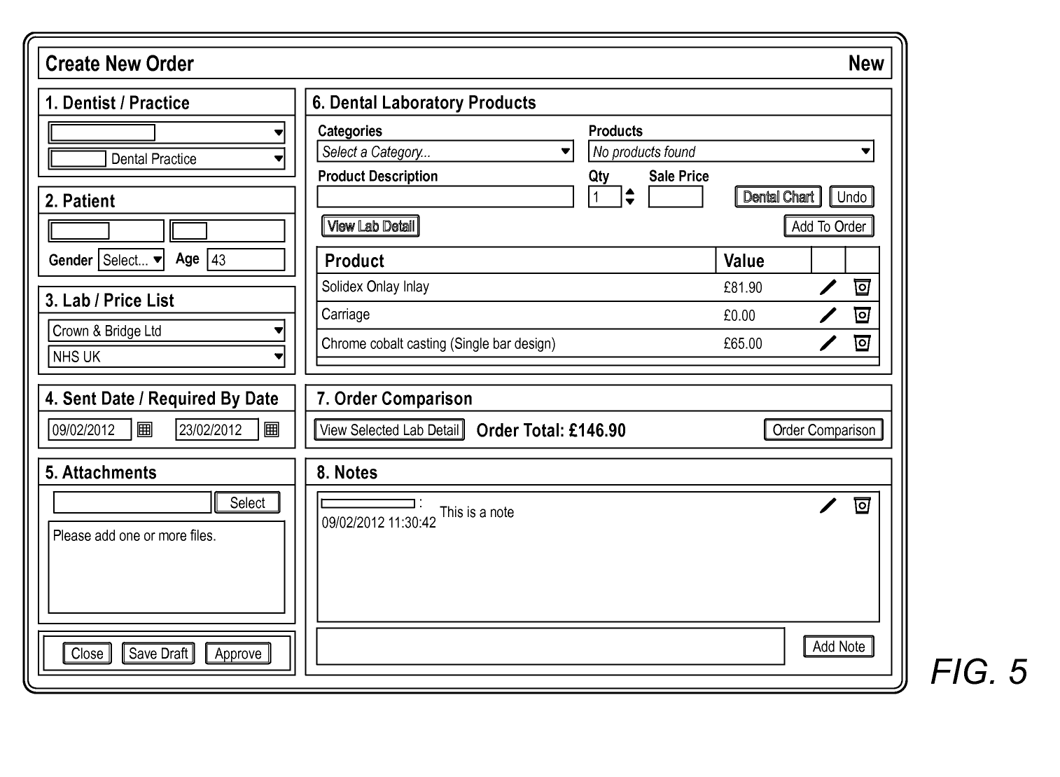

Each laboratory has a price list (PriceList). Each PriceList contains LabProducts (with slightly different names). Each PriceList is priced in a number of different ways. We have built a generic list (coding table above) of product codes to assign LabProduct/s to allow generic labelling (Product/Code/s). We apply the ProductCode/s and Laboratory Description to assign Pricing Rules to the LabProducts based on the following criteria (see FIG. 4 also):

1. Is there a ‘Which Jaw’ application to set.

2. Is there a teeth position application to set which affects the price of a Product?

a. Anterior teeth

b. Premolar teeth

c. Molar teeth

This mainly applies to Crowns and Bridges, but not exclusively.

3. Bridge units are defined as ‘Pontics’ or ‘Retainers’. This allows for different pricing of these two bridge elements.

4. Price types can also apply to 1, 2, or 3 above. i.e.:

a. ‘Standard’ pricing—as when three crowns are made. Then the pricing rule, as applied to crowns, is that, subject to (2.) above, the price is a multiple of the price of a single crown.

b. ‘QTY Range’. This applies to partial dentures, so that a partial denture with 1, 2 or 3 teeth would all be the same price and not three times the product price.

c. ‘Base Price’ & ‘Standard’, as when, for instance, the first tooth added to a denture is one price, but each additional added tooth done at the same time, costs a lot less per tooth added.

d. Combo Only—prices which only apply when used within a combination price. (Not currently required but provides additional flexibility.)

5. ‘Combo’ applications can be any combination of 1, 2, 3 and 4 above and override any prices they are assigned to (if applicable). Combination prices can apply to items in EITHER the upper OR lower jaw or any combination thereof, with the exception of Bridge work. Combination prices for Bridge items must all be within the same jaw.

### Order Process

In one embodiment, the system allows searching to apply a bespoke search criteria in order to compare across multiple laboratories in order to arrive at an order comparison:

Bespoke search criteria may include: (1) price list (2) turnaround times (3) audit feedback (4) product remake rates (5) outsourcing out of UK to non-gdc registered technicians. Additional criteria may also be used.

The process applied to each dentists order entered onto the system may be as follows:

1. Selecting a Product Code

2. Specifying which Jaw they require the product for (if applicable)

3. Specifying which teeth they require (if applicable)

4. Specifying the type of bridge required for each tooth (if applicable)

5. Specifying a quantity (if not already determined by step 3 or 4)

### Price Criteria Comparison

Pricing comparisons may also be made. Below is an example of such price criteria comparisons.

1. Find all Prices for the given ProductCode and PriceList

2. Sort Prices by PriceType, Qty and CombinationPrice Count

3. Loop through all Prices and Assign ProductSelections to matching Prices

4. Repeat Until all ProductSelections have been found or PriceList can't fulfill Order

5. Replace all Prices with qualifying Combination Prices

6. Apply any Base Prices found to Assigned Prices

In some embodiments, items 5 and 6 above may be omitted or optional.

To run a Price Comparison based on a customer Order the following processing steps may occur:

a. Loop through all PriceLists and OrderLines found

b. Calculate (using 1 to 6 above) any Prices for the given ProductCode and PriceList

c. Add all Prices found to Price Comparison options

This process is summarised in the example illustrated by the PseudoCode in Appendix 4.

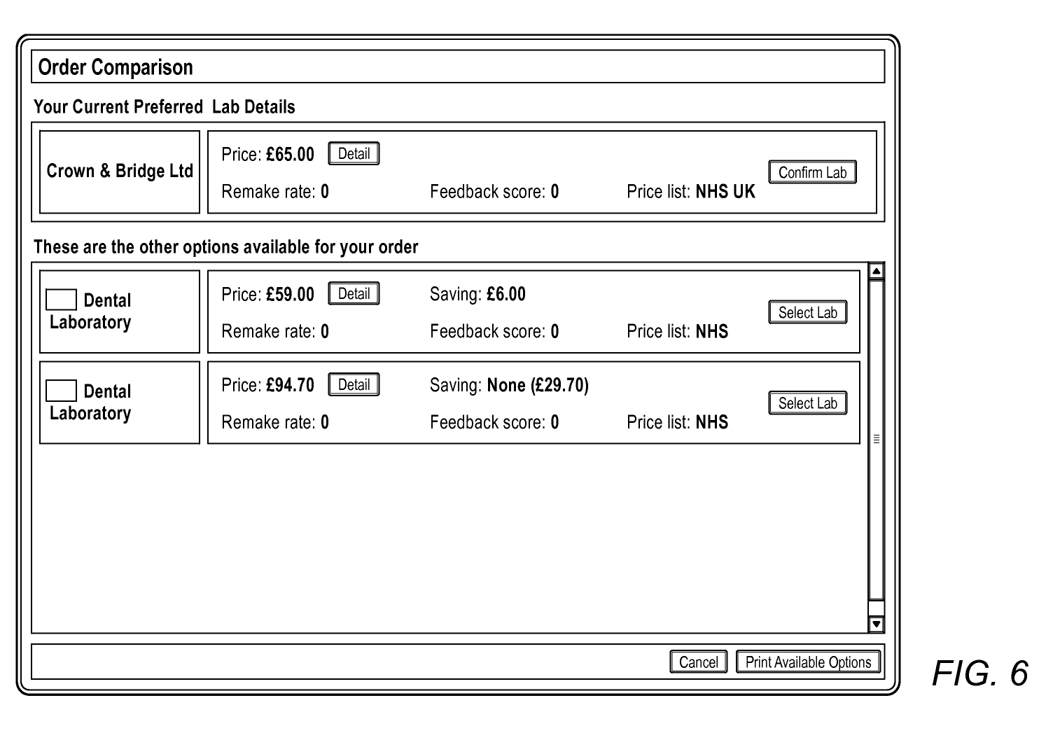

FIG. 5 is an example order form of the system for dentists to complete to make an order for a product.

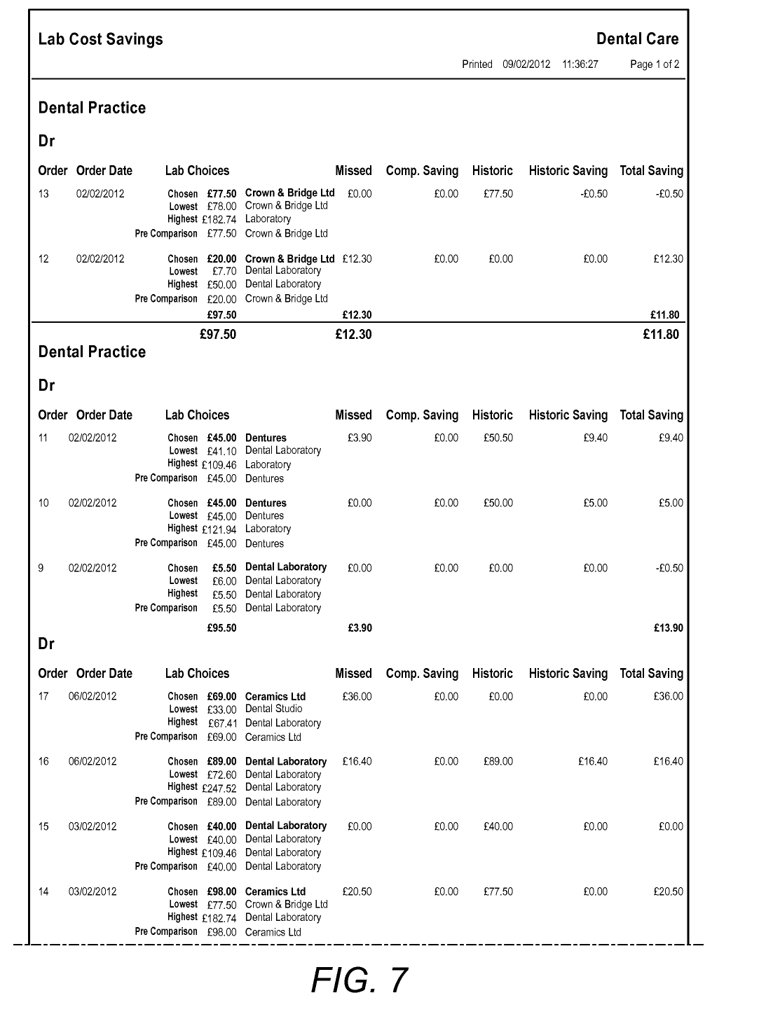

FIG. 6 is an example of obtaining a comparison when ordering a dental laboratory product, i.e. FIG. 6 is an example of the window that shows the different prices available for the order that they are in the process of making. In this example, they have to make this order comparison before they can complete the order.

Other features of an example system of the invention are:

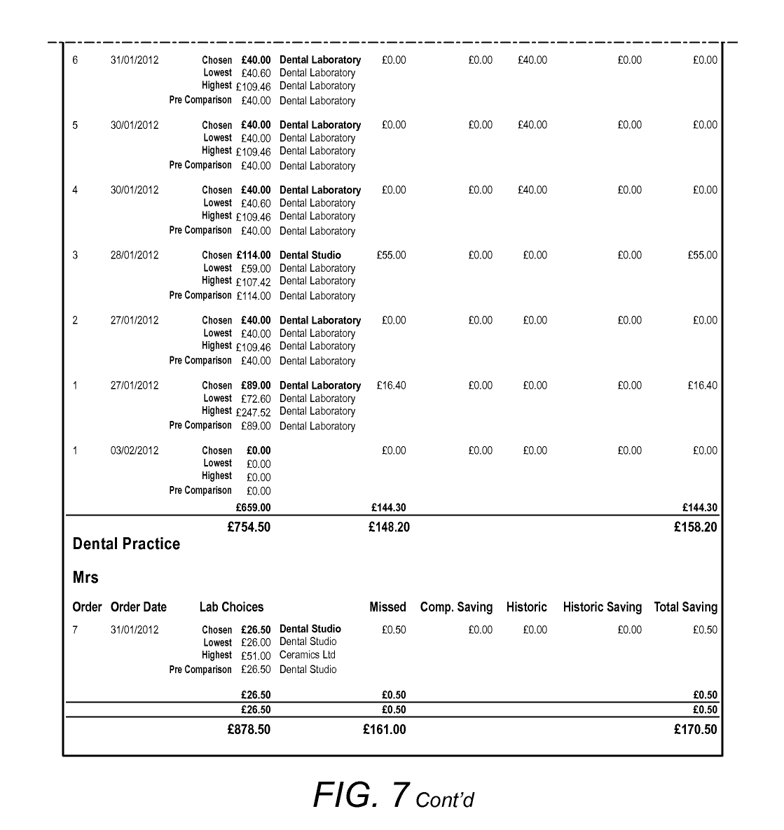

Referring to FIG. 7, this is an example of a report that the group can view the savings that have been made or could have been made for the all the orders made by dentists.

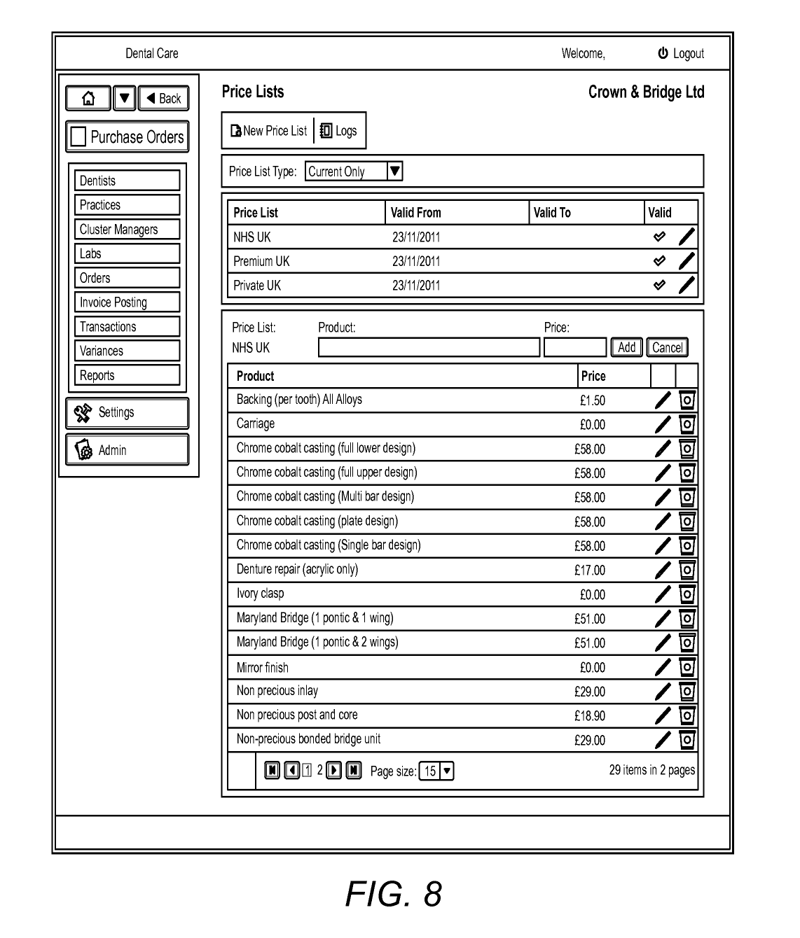

Referring to FIG. 8, this is an example of a form that dentists/practices use to access prices lists for labs.

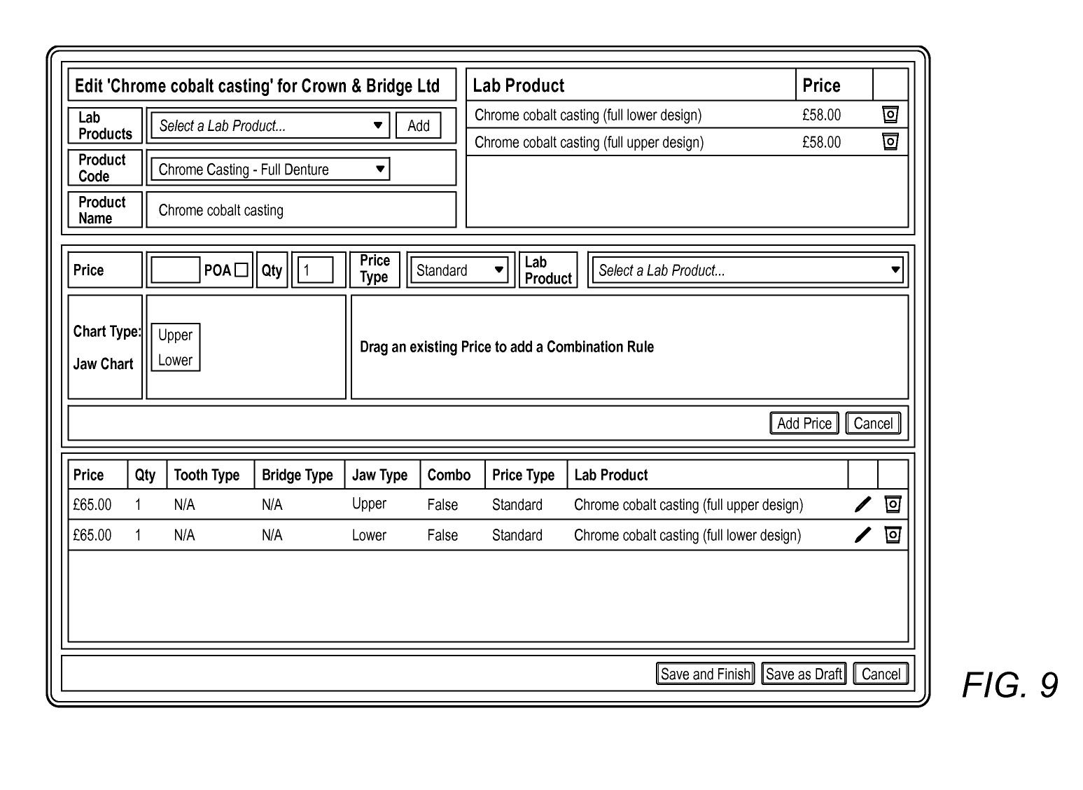

Referring to FIG. 9, this is an example of a form for viewing the products available, for example specifying the comparison rules for any product from any dental laboratory.

The above embodiments are to be understood as illustrative examples of the invention. Further embodiments of the invention are envisaged. Further information of features in accordance with examples of the present invention is given in Appendices 1, 2 and 3 below. Appendix 1 describes features which may be provided in embodiments according to the invention; Appendix 2 is a schematic drawing and written details of an example system for providing a method of the invention; Appendix 3 gives further information of features of an example system for providing a method of the invention.

Further, a system arranged to perform the method of the present invention may also be configured to allow a dental laboratory, currently not in a list of preferred laboratories for supplying dental laboratory products (and from which list a dentist, for example, may compare dental laboratory products from different dental laboratories and select a dental laboratory to order a product from), to apply to be added to the list of preferred laboratories. In this application, a dental laboratory not on the list, but wanting to be added to the list, may provide details of the laboratory, including for example products for supply and their delivery catchment area. On the basis of the application, the system may be configured to compare the provided details against a set of predetermined details held by the system for deciding whether or not to approve the dental laboratory for being added to the preferred list of dental laboratory suppliers. If, based on the comparison, the details meet requirements of the predetermined details, the system approves the application, and the dental laboratory is added to the preferred list. If, however, any of the details provided do not meet the requirements of the predetermined details, the system rejects the application. The application by a dental laboratory may be made by accessing a publicly available application form, via the Internet, for example.

Although the method of the present invention is described in relation to supply of dental laboratory products, it is envisaged that a method according to an embodiment of the present invention may also, or instead, be used in relation to supplying medical laboratory products or veterinary laboratory products, for example, which may be custom made for a patient.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. A method of providing data indicative of options for supply of a dental laboratory product, the method comprising:
receiving data indicative of the dental laboratory product;
retrieving from a data store, or generating, data indicative of a first value of a parameter of the dental laboratory product based on the received data indicative of the dental laboratory product;
retrieving from a data store, or generating, data indicative of a second value of the parameter of the dental laboratory product based on the received data indicative of the dental laboratory product; and
generating data representative of a comparison of the first value with the second value.

2. The method of claim 1, wherein generating the data includes comparing the first value with the second value according to a comparison criteria, and generating data indicative of a preferred option for supply of the dental laboratory product.

3. The method of claim 2, wherein the generated data comprises data representative of a list comprising the first and second values.

4. The method of claim 2, wherein the comparison criteria comprises identifying the lowest of the first and second value.

5. The method of claim 4, wherein the parameter is price, and the first value relates to a first dental laboratory product supplier and the second value relates to a different, second dental laboratory product supplier.

6. The method of claim 5, wherein the received data indicative of the dental laboratory product comprises data indicating a dental laboratory product type.

7. The method of claim 6, wherein the dental laboratory product type is one of a jaw product, a tooth product and a bridge product.

8. The method of claim 7, wherein the dental laboratory product type relates to one of: an upper jaw position, a lower position, any jaw position, an anterior tooth position, a premolar tooth position, a molar tooth position, a combination of any of the aforesaid tooth positions, any tooth position, a pontic bridge type, a retainer bridge type and any bridge type.

9. The method of claim 8, wherein the retrieved data indicative of the first value is generated using a first calculation algorithm to convert data indicative of the first value from a first data format to a standard data format, and the retrieved data indicative of the second value is generated using a different, second calculation algorithm to convert data indicative of the second value from a different, second data format to the standard data format.

10. The method of claim 9, further comprising:
selecting a dental laboratory for supply of the dental laboratory product the generated data representative of a comparison of the first value with the second value, data indicative of a value of the dental laboratory product from a standard data format to a different data format recognizable by the selected dental laboratory; and
transmitting data indicative of the dental laboratory product to the selected dental laboratory, the transmitted data including the value being in said different data format.

11. The method of claim 2, further comprising using said data indicative of a preferred option for supply of the dental laboratory product to identify a preferred supplier of the dental laboratory product.

12. The method of claim 11, further comprising:
retrieving from a data store or generating data indicative of a first value of at least one further parameter of the dental laboratory product based on the received data indicative of the dental laboratory product;
retrieving from a data store or generating data indicative of a second value of the at least one further parameter of the dental laboratory product based on the received data indicative of the dental laboratory product,
wherein the generating the data indicative of the preferred option for supply of the dental laboratory product includes comparing the first value with the second value of the at least one further parameter.

13. The method of claim 12, wherein the at least one further parameter includes at least one of a quality of a dental laboratory product from a supplier, a delivery time of a dental laboratory product from a supplier, and a delivery area of a dental laboratory product supplier.

14. The method of claim 13, wherein said generating the data indicative of the preferred option includes preferentially weighting one of the parameter and the at least one further parameter such that the data indicative of the preferred option is generated according to the preferential weighting.

15. The method of claim 14, wherein the received data indicative of the dental laboratory product includes data identifying the dental laboratory product.

16. The method of claim 15, wherein the dental laboratory product is custom-made for a patient.

17. A method of generating data indicative of a parameter of a dental laboratory product, the method comprising:
generating, by a processor, a first value of the parameter of the dental laboratory product using a first calculation algorithm to convert data indicative of the first value from a first data format to a standard data format,
generating, by the processor, a second value of the parameter of the dental laboratory product using a different, calculation algorithm to convert data indicative of the second value from a different, second data format to the standard data format.

18. The method of claim 17, wherein the parameter is price.

19. The method of claim 18, wherein the first data format is provided by a first supplier of the dental laboratory product and the second data format is provided by a different, second supplier of the dental laboratory product.

20. A method of providing data indicative of options for supply of a medical or veterinary laboratory product, the method including:
a) receiving data indicative of the medical or veterinary laboratory product;
b) on the basis of said data received in step a), retrieving from a data store, or generating, data indicative of a first value of a parameter of the medical or veterinary laboratory product;
c) on the basis of said data received in step a), retrieving from a data store, or generating, data indicative of a second value of the parameter of the medical or veterinary laboratory product; and
d) generating data representative of a comparison of the first value with the second value.
